(19) United States
(12) Patent Application Publication
(10) Pub. No.: US 2026/0234084 A1
(43) Pub. Date: Aug. 13, 2026

(54) PROCESS FOR HYDROGENATING A SUBSTRATE

(71) Applicant: JOHNSON MATTHEY DAVY TECHNOLOGIES LIMITED, London (GB)

(72) Inventor: Adrian BACKES, London (GB)

(21) Appl. No.: 19/470,914

(22) PCT Filed: May 14, 2024
(86) PCT No.: PCT/GB2024/051245

§ 371 Date: Sep. 30, 2025

(30) Foreign Application Priority Data
May 15, 2023 (GB) 2307175.6

(51) Int. Cl. C07C 29/141; B01J 8/02

(52) U.S. Cl. CPC C07C 29/141; B01J 8/0278

(57) ABSTRACT

A process for hydrogenating a substrate in a hydrogenation reactor to provide a liquid product, said process comprising: (a) supplying to the hydrogenation reactor; (i) fresh substrate; (ii) fresh hydrogen gas; (ii) recycled liquid product; (iii) recycled hydrogen gas; (c) collecting liquid product in a liquid product stream from an outlet of the hydrogenation reactor; (d) collecting liquid product in a liquid product recycle stream and returning at least portion to the hydrogenation reactor as the recycled liquid product; (e) collecting hydrogen gas from an outlet of the hydrogenation reactor as a hydrogen gas recycle stream and returning at least a portion to the hydrogenation reactor as the recycled hydrogen gas; wherein the hydrogen gas recycle stream is passed through an eductor before it is returned to the hydrogenation reactor.

### FIELD OF THE INVENTION

The present invention relates to a process and apparatus for hydrogenating a substrate. More particularly it relates to a process for hydrogenating an aldehyde, which may be unsaturated, to provide a saturated alcohol.

### BACKGROUND

Conventional liquid phase hydrogenation reactors, such as those used to produce so-called “Oxo” alcohols by hydrogenation of aldehydes formed via hydroformylation of an alkene and optional aldolisation as described, for example, in U.S. Pat. Nos. 4,148,830 and 5,087,763, operate with only a very small excess of hydrogen gas to minimise vent losses of hydrogen. Such reactors also operate with a large liquid recycle in order to minimise the reactor adiabatic temperature rise.

Running with such a small hydrogen excess makes the hydrogenation reactor vulnerable to localised depletion in hydrogen with subsequent catalytic underperformance and increased by-product formation. The reactor is also more vulnerable to underperformance in the event of less than complete mixing of liquid recycle and fresh aldehyde feed.

The present invention seeks to address the problem of effectively balancing hydrogen losses with optimal reactor performance whilst not greatly increasing costs and maintenance requirements on a plant.

### SUMMARY OF THE INVENTION

The inventors have found, in particular, that using the large liquid recycle flow as the motive fluid in an eductor to drive a circulation of hydrogen around the hydrogenation reactor increases the availability of hydrogen in the reactor, which is borne out in improved substrate conversion and reduced by-product formation. Therefore, the performance of the reactor can be improved in terms of increased substrate conversion and reduced by-product formation, without increasing the amount of hydrogen provided to the reactor and in turn increasing vent losses of hydrogen. This is achieved without the increased cost and maintenance requirement of a dedicated recycle compressor for the hydrogen recycle.

Accordingly, the present invention provides a process for hydrogenating a substrate in a hydrogenation reactor to provide a liquid product, said process comprising:
(a) supplying to the hydrogenation reactor;
   (i) fresh substrate;
   (ii) fresh hydrogen gas;
   (ii) recycled liquid product;
   (iii) recycled hydrogen gas;
(b) collecting liquid product in a liquid product stream from an outlet of the hydrogenation reactor;
(c) collecting liquid product in a liquid product recycle stream and returning at least a portion, preferably substantially all, to the hydrogenation reactor as the recycled liquid product;
(d) collecting hydrogen gas from an outlet of the hydrogenation reactor as a hydrogen gas recycle stream and returning at least a portion, preferably substantially all, to the hydrogenation reactor as the recycled hydrogen gas;
wherein the hydrogen gas recycle stream is passed through an eductor before it is returned to the hydrogenation reactor; and
wherein the motive fluid in the eductor is the fresh substrate, the liquid product recycle stream or a combination of the fresh substrate and the liquid product recycle stream, or the motive fluid is the fresh hydrogen gas.

Also provided is an apparatus for hydrogenating a substrate, said apparatus comprising;
(a) a hydrogenation reactor configured to hydrogenate the substrate and provide a liquid product;
(b) means for supplying fresh substrate to the hydrogenation reactor;
(c) means for supplying fresh hydrogen gas to the hydrogenation reactor;
(d) means for collecting a liquid product stream from an outlet of the hydrogenation reactor;
(e) means for collecting liquid product as a liquid product recycle stream;
(e) means for returning the liquid product recycle stream to the hydrogenation reactor;
(f) means for collecting hydrogen gas from an outlet of the hydrogenation reactor as a hydrogen gas recycle stream,
(g) means for returning the hydrogen gas recycle stream to the hydrogenation reactor;
(f) an eductor configured to receive the hydrogen gas recycle stream before it is returned to the hydrogenation reactor;
wherein the eductor is configured to use the fresh substrate, the liquid product recycle stream or a combination of the fresh substrate and the liquid product recycle stream as the motive fluid.

Preferably the substrate is an aldehyde or acrolein, being hydrogenated to an alcohol. In a preferred embodiment the motive fluid in the eductor is the fresh substrate, the liquid product recycle stream or a combination of the fresh substrate and the liquid product recycle stream. Most preferably the motive fluid in the eductor comprises the liquid product recycle stream. Preferably the ratio of the liquid product recycle stream to the fresh substrate (i.e. mass flowrate of liquid product recycle stream: mass flowrate of fresh substrate) is at least 10:1, preferably at least 20:1. In such an embodiment, the flow of hydrogen through the hydrogenation reactor is preferably at least 120%, more preferably at least 130% of the stoichiometric flow of hydrogen required to hydrogenate the flow of substrate through the hydrogenation reactor. Such flowrates might also be termed a 20%, preferably 30%, excess of hydrogen. To use such an excess of hydrogen in the hydrogenation reactor without a recycle would involve a costly purging of unreacted hydrogen. However, to compress such an excess of hydrogen in a recycle using a compressor may be uneconomical. The invention advantageously uses the large liquid product recycle flow, which is advantageous from a heat removal perspective, to drive a hydrogen gas recycle to provide a significant hydrogen excess in an economical way. That in turn advantageously reduces the risk of hydrogen starvation in the hydrogenation reactor. This advantageous combination of a large liquid product recycle and large hydrogen gas recycle enabled by an eductor driven by the large liquid product recycle may be particularly advantageous in the hydrogenation of aldehyde or acrolein to alcohol as part of an oxo alcohols process. In some embodiments the process further comprises a polishing reactor, which may be a separate reactor or a separate catalyst bed within the same reactor. The liquid product stream from the hydrogenation reactor is fed to the polishing reactor along with a hydrogen stream, which preferably comprises fresh hydrogen. A hydrogen excess is also desirable in the polishing reactor, for example to drive high conversion, and the flow of hydrogen through the polishing reactor is preferably at least 105%, more preferably at least 110% of the stoichiometric flow of hydrogen required to hydrogenate the flow of substrate through the reactor. This could represent a further loss of unreacted hydrogen, but according to the invention the hydrogen gas exiting the polishing reactor is preferably combined with the hydrogen gas collected from the hydrogenation reactor to form part of the hydrogen gas recycle stream received by the eductor. In that way, the large liquid product recycle on the hydrogenation reactor is advantageously used not only to drive an excess of hydrogen gas through the hydrogenation reactor but also an excess of hydrogen gas through the polishing reactor.

### BRIEF DESCRIPTION OF THE DRAWINGS

FIG. 1 is a schematic diagram of a conventional liquid hydrogenation process.

FIG. 2 is a schematic diagram of a process according to the invention.

FIG. 3 is a schematic diagram of a further process according to the invention.

FIG. 4 is a schematic diagram of an eductor as used in a process according to the invention.

FIG. 5 is a table of data demonstrating the relationship between the amount of hydrogen passing through a reactor, catalytic activity (in terms of aldehyde slip) and by-product make in the hydrogenation of butyraldehyde.

### DETAILED DESCRIPTION OF THE INVENTION

Hydrogenating a substrate means adding molecular hydrogen across one or more double bonds in a molecule to provide the corresponding single bond(s). The nature of the double bond is not particularly limited, examples being hydrogenating a carbon-carbon double bond to provide a carbon-carbon single bond as well as hydrogenating a carbon-oxygen double bond to provide a carbon-oxygen single bond.

The substrate may be any substrate containing a double bond which can be hydrogenated using gaseous hydrogen in a liquid phase hydrogenation to produce a liquid hydrogenation product. In particular, those substrates for which the hydrogenation reaction requires a large liquid recycle to minimise the reactor adiabatic temperature rise. Suitably, the substrate may be an aldehyde and the product an alcohol. The aldehyde may be a saturated aldehyde which is typically linear and the product the corresponding saturated alcohol, or the aldehyde may be an α,β-unsaturated aldehyde e.g. a 2-alkylalkenal and the product the corresponding saturated alcohol e.g. a 2-alkylalkanol. Saturated aldehydes are typically C₃ to C₂₀, suitably C₃ to C₁₅, and unsaturated aldehydes are typically C₄ to C₂₀, suitably C₄ to C₁₅. Example aldehyde substrates and alcohol products include butyraldehyde and butanol; 2-ethyl hex-2-enal and 2-ethyl hexanol; 2-propyl hept-2-enal and 2-propyl heptanol; isononyl aldehyde and isononyl alcohol. Fresh substrate is fed to the system as a liquid stream which may enter the eductor directly, enter the hydrogenation reactor directly, or first combine with the liquid product recycle stream either upstream or downstream of the eductor. The feed point of the fresh substrate will depend largely on the motive fluid being used in the eductor.

The hydrogenation reactor may be operated under any suitable conditions. A catalyst will generally be used. Any suitable catalyst may be used. Generally, particularly in the case of aldehydes, including α,β-unsaturated aldehydes, the active component of the catalyst will be based on metals from Groups VI to XI. Suitable examples include copper, nickel, manganese, zinc, cobalt, palladium, ruthenium and iron. The catalyst may be supported. Any suitable support may be used. Suitable supports include alumina, silica, aluminasilicates, titania, zirconia or diatomaceous earth. A particularly suitable catalyst may be a supported copper chromite or copper alumina catalyst, particularly in the case of aldehydes, including α,β-unsaturated aldehydes. The catalyst may also, for example, include a promoter to enhance selectivity.

The hydrogenation is carried out in the liquid phase. Any suitable configuration may be used and the reactor may be operated under any suitable conditions. The particular conditions selected will depend on the catalyst chosen. For example, particularly in the case of aldehydes, including α,β-unsaturated aldehydes, the hydrogenation may be carried out at a temperature of from about 100° C. to about 200° C. and at a pressure of from atmospheric to about 15 MPa. In one arrangement the hydrogenation may be carried out as downflow over packed beds of catalyst.

Where a nickel catalyst is used in a liquid phase reaction, the temperature may be below about 150° C. at a pressure of from about 1 to about 3 MPa, particularly in the case of aldehydes, including α,β-unsaturated aldehydes. Where a copper chromite catalyst is used in a liquid phase reaction the temperature may be from about 50° C. to about 200° C. at a pressure of from about 0.1 MPa to about 5 MPa, for example from about 0.5 MPa to about 5 MPa, particularly in the case of aldehydes, including α,β-unsaturated aldehydes.

The recycle of liquid product is carried out to remove the heat of reaction. As a skilled person understands, the heat release during the reaction will depend on the substrate. For example, in the case of an aldehyde substrate, the greater the number of carbons in the aldehyde the lower the heat release per unit mass. Typically, the temperature rise will be kept below about 50° C. The recycle ratio of liquid product to fresh substrate is typically from about 2:1 to about 60:1 on a mass basis, suitably from about 5:1 to about 40:1. In some embodiments the recycle ratio of liquid product to fresh substrate is preferably from about 10:1, or more preferably from about 20:1, to about 100:1, or more preferably to about 60:1, or yet more preferably to about 40:1, on a mass basis. Typically, the liquid product recycle stream is passed through a cooler before any recycled liquid product is returned to the hydrogenation reactor. The cooler may be, for example, a heat exchanger in which the removed heat is used to raise steam. The liquid product recycle stream will typically pass through a recycle pump before it passes through the eductor. When the liquid product recycle stream is combined with the fresh substrate to provide the motive fluid, liquid product recycle stream will typically pass through the recycle pump before it is combined with the fresh substrate.

As is generally known, an eductor is a pump which uses the Venturi effect to pump a fluid in an enclosed line using a motive fluid. The motive fluid provides the pumping energy. After entering the eductor, the motive fluid flows through a converging section into a constricted throat which causes a velocity increase and a pressure decrease. After exiting the throat, the motive fluid enters a diverging section, or nozzle, which causes a velocity decrease and a pressure increase, this creates a region of low pressure which is used to draw in and pump the other fluid. The motive fluid and the other fluid exit the eductor, via the nozzle as a single stream. The present invention particularly uses the liquid product recycle stream, which is typically large in liquid phase hydrogenation, to pump the hydrogen gas recycle and more effectively use the hydrogen in the system. Accordingly, the motive fluid is preferably the liquid product recycle stream, or a combination of the fresh substrate and the liquid product recycle stream. However, the fresh substrate feed may be large enough to act effectively as the motive fluid alone and so alternatively the fresh substrate feed may be used as the motive fluid. Alternatively, in some instances the fresh hydrogen gas can be used as the motive fluid. This can be advantageous when a hydrogen feed having a relatively high pressure is used and as such can tolerate at least a tangible drop in pressure across before it is supplied to the reactor.

The stream used as the motive fluid must be at a high enough pressure to tolerate at least a tangible drop in pressure across the eductor. The motive fluid pressure drop across the eductor is not particularly limited and will depend on the nature and quantity of the motive fluid as well as the particular reactor design. A typical pressure drop where the recycle liquid is the fresh substrate, the liquid product recycle stream or a combination of the fresh substrate and the liquid product recycle stream may be in the range of about 0.02 to about 1.0 MPa, suitably about 0.05 to about 0.40 MPa. The flow rate of the liquid motive fluid will depend on the nature and scale of the hydrogenation reaction, and the reactor design

The hydrogen gas recycle stream will typically be between about 0.1 and about 5 times the stoichiometric requirement of hydrogen, suitably between about 0.2 and about 1 times the stoichiometric requirement of hydrogen. There will also be a vent in the hydrogenation reactor, for the purpose of maintaining the desired partial pressure of hydrogen in the reactor, and to purge inerts from the system. This vent may be taken directly from the reactor, or from the hydrogen gas recycle stream. This vent will be no larger in flow than the vent from a hydrogenation reactor which does not employ the hydrogen gas recycle of the present invention. A typical vent purge of hydrogen may be between about 1% and 30% of the stoichiometric requirement of hydrogen but is not particularly limited and can be influenced by hydrogen gas feed impurities and also by-product gas make. It is an advantage of the invention that higher catalyst activity and lower by-product make is achieved without increasing the equivalents of hydrogen supplied to the reactor, which in turn increases the amount of hydrogen which must be vented and thus lost from the system. Typically, the volume ratio of liquid exiting the reactor to gas exiting the reactor will be less than 20:1, suitably less than 10:1, preferably less than 5:1. The volume ratio will typically be greater than 0.5:1. Gas exiting the reactor means via all outlets including vent and recycle streams. Liquid exiting the reactor means via all outlets including product outlet and recycle streams. The majority of the gas exiting the reactor will of course be via the recycle stream, which leads to the benefits of the invention without an increased vent of hydrogen.

The fresh substrate, recycled liquid product and recycled hydrogen gas are typically supplied to the hydrogenation reactor though a single inlet as one stream. However, alternative ways of supplying are envisaged. For example, when the motive fluid is a combination of the fresh substrate and the liquid product recycle stream, a stream which comprises the fresh substrate, the liquid product recycled stream and the recycled hydrogen gas will exit the eductor. This stream can be supplied directly to the reactor, or can pass through a disengagement pot in which hydrogen gas is separated from the stream and supplied to the hydrogenation reactor via a separate inlet. When only one of the fresh substrate and the liquid product recycle stream is the motive fluid, the other stream can be combined with the stream which exits the eductor before supplying to the hydrogenation reactor, or it can be supplied via a separate inlet. The invention is achieved providing that the hydrogen gas recycle stream and a motive fluid as defined herein pass through the eductor, and the fresh substrate, recycled liquid product and recycled hydrogen gas are supplied to the hydrogenation reactor. The fresh hydrogen gas is typically fed to the hydrogenation reactor via a separate inlet.

In the process of the invention, when the substrate is an α,β-unsaturated aldehydes, the majority of the α,β-unsaturated aldehyde will be hydrogenated. However, some products of partial hydrogenation may also be formed. For example, in the case of hydrogenation of 2-alkylalkenals to provide 2-alkylalkanols, partial hydrogenation products such as 2-alkylalkanals and 2-alkylalkenols may form. Also, heavies may form i.e. materials which have a higher molecular weight and/or boiling point than the desired product alcohol. Accordingly, the process of the present invention may include additional distillation and polishing steps to provide the desired product in a high degree of purity, for example as disclosed in WO2018/069714 in the case of the production of 2-alkylalkanols, or WO2019/197831 in the case of the co-production of alcohols such as butanol and 2-alkylalkanols.

The present invention may be particularly advantageous in a process for the production of oxo alcohols. Thus, in a particularly preferred aspect of the invention there is provided a process for the production of oxo alcohols, the process comprising hydroformylating a mixture of an olefin and syngas in the presence of hydroformylation catalyst to produce an aldehyde, separating the aldehyde from the catalyst by vaporisation of the aldehyde from the catalyst, and hydrogenating the aldehyde to an alcohol, wherein the hydrogenating comprises (a) supplying to the hydrogenation reactor: (i) the aldehyde; (ii) fresh hydrogen gas; (iii) recycled liquid product comprising the alcohol; and (iv) recycled hydrogen gas; (b) collecting liquid product comprising the alcohol in a liquid product stream from an outlet of the hydrogenation reactor; (c) collecting liquid product comprising the alcohol in a liquid product recycle stream and returning at least portion to the hydrogenation reactor as the recycled liquid product; and (d) collecting hydrogen gas from an outlet of the hydrogenation reactor as a hydrogen gas recycle stream and returning at least a portion to the hydrogenation reactor as the recycled hydrogen gas; wherein the hydrogen gas recycle stream is passed through an eductor before it is returned to the hydrogenation reactor; and wherein the motive fluid in the eductor is the aldehyde, the liquid product recycle stream or a combination of the aldehyde and the liquid product recycle stream, or the motive fluid is the fresh hydrogen gas, Most preferably the motive fluid comprises the liquid product recycle stream. In a further particularly preferred aspect of the invention there is provided a process for the production of oxo alcohols, the process comprising hydroformylating a mixture of an olefin and syngas in the presence of hydroformylation catalyst to produce an aldehyde, separating the aldehyde from the catalyst by vaporisation of the aldehyde from the catalyst, subjecting the aldehyde to aldolization to form an acrolein and hydrogenating the acrolein to an alcohol, wherein the hydrogenating comprises (a) supplying to the hydrogenation reactor: (i) the acrolein; (ii) fresh hydrogen gas; (iii) recycled liquid product comprising the alcohol; and (iv) recycled hydrogen gas; (b) collecting liquid product comprising the alcohol in a liquid product stream from an outlet of the hydrogenation reactor; (c) collecting liquid product comprising the alcohol in a liquid product recycle stream and returning at least portion to the hydrogenation reactor as the recycled liquid product; and (d) collecting hydrogen gas from an outlet of the hydrogenation reactor as a hydrogen gas recycle stream and returning at least a portion to the hydrogenation reactor as the recycled hydrogen gas; wherein the hydrogen gas recycle stream is passed through an eductor before it is returned to the hydrogenation reactor; and wherein the motive fluid in the eductor is the acrolein, the liquid product recycle stream or a combination of the acrolein and the liquid product recycle stream, or the motive fluid is the fresh hydrogen gas, In either of the particularly preferred embodiments, the olefin may be obtained from Fischer Tropsch reactions, methanol-to-olefin process, from a refinery, from sustainable sources or from any other source of olefin. The syngas may, for example, be obtained from reforming (of coal, natural gas, or any other suitable feedstock) or gasification. The syngas may also be obtained from sustainable feedstocks. An example is capturing carbon dioxide and producing hydrogen from electrolysis, or another source, before using the reverse water gas shift reaction to convert the carbon dioxide and hydrogen to syngas. Where there are contaminants present in the olefin and/or the syngas these can be removed in a feedstock purification section.

Hydroformylation is the reaction of carbon monoxide with olefin to form aldehyde. The aldehyde has one more carbon that the olefin. Hydroformylation is typically carried out using a homogeneous rhodium catalyst with an organophosphorus ligand, such as those listed in U.S. Pat. Nos. 4,769,498, 4,885,401, 5,113,022, 5,202,297, 5,235,113, 4,668,651, 4,748,261, 5,254,741, 5,391,801, 5,059,710, 3,527,809, 4,283,562, 4,400,548, 4,482,749, 4,599,206, 4,716,250, 4,717,775, 4,731,486, 4,737,588 or WO8001690. Typically, the hydroformylation reaction is carried out in two or more reactors. Aldehyde product and the rhodium catalyst are passed from the reactors to a catalyst recovery section which separates the aldehyde product from the rhodium catalyst and its solvent. The solvent may typically be the aldehyde, heavies formed in the reaction or any other suitable solvent. Examples of techniques to separate the rhodium catalyst, or to stabilise the rhodium catalyst can be found in U.S. Pat. Nos. 4,774,361, 5,874,640, 5,892,119, 6,090,987, 6,294,700, 6,100,432, 5,114,473, 4,148,830 and 4,247,486.

The aldehyde will contain a mix of normal and iso aldehyde. The aldehyde may be hydrogenated to both normal and iso alcohol, or aldolized and hydrogenated to a mixture of branched alcohols, such as C10 alcohols in the form of a mixture of 2-propylheptanol isomers. However, the aldehyde is preferaby sent to a distillation column to substantially separate the normal and iso aldehyde.

Downstream of the distillation column, normal aldehyde, such as n-butyraldehyde, is preferably then either hydrogenated to normal alcohol, such as n-butanol, or passed to an aldolization section. The iso-aldehyde may be used as a product or may, in some cases, such as when 2-propylheptanol is being produced, be passed with some or all of the normal aldehyde to an aldolization section.

In aldolization the aldehyde reacts to form an acrolein (also known as an alkenal), which is preferably then hydrogenated to an alcohol. Examples include aldolization of n-butyraldehyde to form ethyl-propyl-acrolein (also known as 2-ethylhexenal), which is then hydrogenated to 2-ethylhexanol, or aldolization of valeraldehyde to propyl-butyl-acrolein (also known as 2-propylheptenal), which is then hydrogenated to 2-propylheptanol.

The hydrogenation reactor, whether of the normal-or iso-aldehyde coming from the distillation column, or of the acrolein from the aldolization, preferably comprises: a primary catalyst bed having an inlet end and an outlet end; means for supplying a primary feed stream to the inlet end of the primary catalyst bed, the primary feed stream comprising the fresh feed and the recycled liquid product; a secondary catalyst bed having an inlet end and an outlet end, the secondary catalyst bed extending substantially vertically through the primary catalyst bed; means for supplying a secondary feed stream to the inlet end of the secondary catalyst bed, the secondary feed stream comprising the recycled liquid product; means for collecting the liquid product from the outlet end of the primary catalyst bed and recycling at least a portion of the liquid product as the recycled liquid product to the inlet end of the primary catalyst bed and secondary catalyst bed; a separating wall between the primary catalyst bed and secondary catalyst bed; means for supplying a primary gas stream comprising the fresh hydrogen gas and/or the recycled hydrogen gas only to the inlet end of the primary catalyst bed; and means for supplying a secondary gas stream comprising the fresh hydrogen gas and/or the recycled hydrogen gas only to the inlet end of the secondary catalyst bed. In such a reactor, the primary catalyst bed may be the hydrogenation reactor described herein and the secondary catalyst bed may be the polishing reactor described herein.

The product alcohols are preferably refined, typically in one or more distillation columns, to achieve desired purities. Examples of possible refining schemes are disclosed in WO2018/069714 and WO2019/197831.

The present invention may be particularly advantageous in the production of 2-alkylalkanol, for example 2-ethylhexanol or 2-propylheptanol and preferably 2-ethylhexanol. Thus, the olefin and syngas may be reacted via hydroformylation to produce normal-and iso-aldehyde and the normal-and iso-aldehyde separated in a distillation column. The normal-aldehyde is preferably aldolized to produce 2-alkylalkenal and the 2-alkylalkenal is preferably hydrogenated to 2-alkylalkanol using the process described above. The iso-aldehyde is typically either hydrogenated to iso-alkanol using the process described above or sold as iso-aldehyde. In some processes, only some of the normal-aldehyde is used for 2-alkylalkanol production and some of the normal-aldehyde is hydrogenated to normal-alkanol using the process described above. As a specific example, propene and syngas may be hydroformylated to form a mix of iso-butyraldehyde and normal-butyraldehyde. Following separation of the iso-butyraldehyde and normal-butyraldehyde in a distillation column at least some of the normal-butyraldehyde is aldolized to 2-ethylhexenal, which is then hydrogenated to 2-ethylhexanol. The iso-butyraldehyde may be hydrogenated to iso-butanol or sold as iso-butyraldehyde. Some of the normal-butyraldehyde may be hydrogenated to normal-butanol. The present invention will now be described, by way of example, with reference to the accompanying figures. It will be understood by those skilled in the art that the drawings are diagrammatic and that further items of equipment such as temperature sensors, pressure relief valves, control valves, flow controllers, level controllers, and the like may be required in a commercial plant. The provision of such ancillary items of equipment forms no part of the present invention and is in accordance with conventional chemical engineering practice.

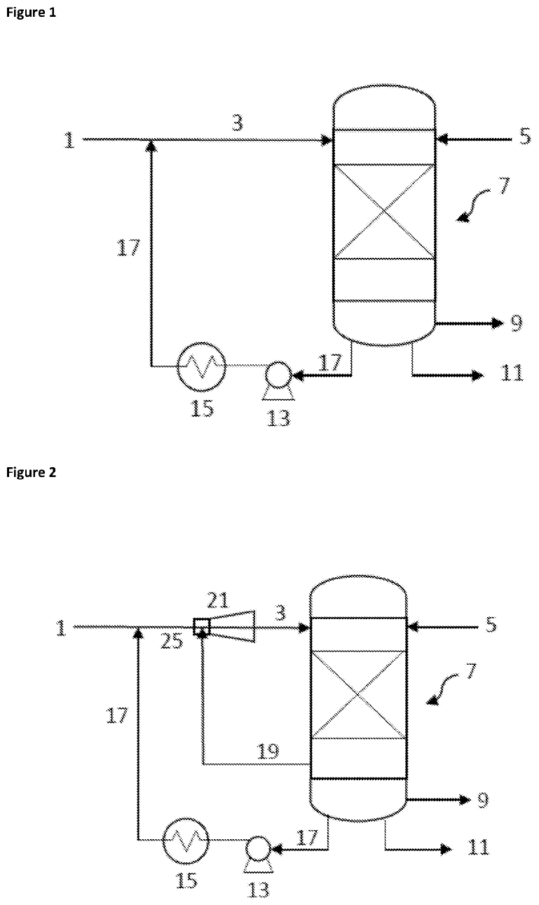

FIG. 1 illustrates a conventional hydrogenation process, for example for hydrogenating an aldehyde, which employs a liquid recycle. Fresh hydrogen gas is supplied as stream 5 to hydrogenation reactor 7, which contains a suitable hydrogenation catalyst, which may be a copper catalyst in the case of an aldehyde. Also supplied to reactor 7 via single stream 3 is a combination of fresh substrate from stream 1 and liquid recycle 17. A liquid stream 11 which comprises liquid product, the corresponding alcohol in the case of an aldehyde substrate, is taken from an outlet at the bottom of the reactor. A liquid recycle stream 17 is also taken from the bottom of the reactor. This is passed through recycle pump 13 and heat exchanger 15, which contains a cooling fluid and takes heat of reaction from the system. The liquid recycle stream is combined with the fresh substrate stream 1 after it has been cooled. A gas purge 9 is also taken from reactor 7, which contains inerts and excess hydrogen. This is typically sent to vent. The vent flow is sufficient to maintain an adequate partial pressure of hydrogen in the reactor such that a near stoichiometric amount of hydrogen is present in the reactor and hydrogen is not wasted, i.e. the flow of hydrogen in the vent is a small percentage of the stoichiometric requirement of hydrogen. For example, the volume ratio of liquid exiting the reactor to gas exiting via vent stream 9 can be, for example, greater than 50:1. However, as described above such a level of hydrogen in the reactor can lead to underperformance in terms of lower catalyst activity and higher by-product make.

FIG. 2 illustrates a process according to the invention in which a hydrogen gas recycle stream 19 is taken from the reactor 7. Recycle stream 19 is passed through liquid motive eductor 21 in which the motive fluid stream 25 is the combination of fresh substrate feed 1 and liquid recycle stream 17. The combination of fresh substrate, liquid recycle stream and hydrogen gas recycle which exits the eductor 21 is fed into the reactor 7 as stream 3. The system is effectively using the motive power from liquid recycle pump 13, which is necessary for the typically large liquid recycle required in such a system (an example being around 20:1 by mass for an aldehyde hydrogenation) to pump the hydrogen gas recycle via the action of the eductor. There will also be a vent 9 to purge inerts from the system, in which some hydrogen will be vented to maintain the desired partial pressure of hydrogen in the reactor. In this system, the volume ratio of liquid exiting the reactor to gas exiting via vent stream 9 is no greater than in the system shown in FIG. 1. However, the catalyst performance is higher, primarily due to a higher availability of hydrogen in the reactor, which is achieved without the addition of a recycle compressor.

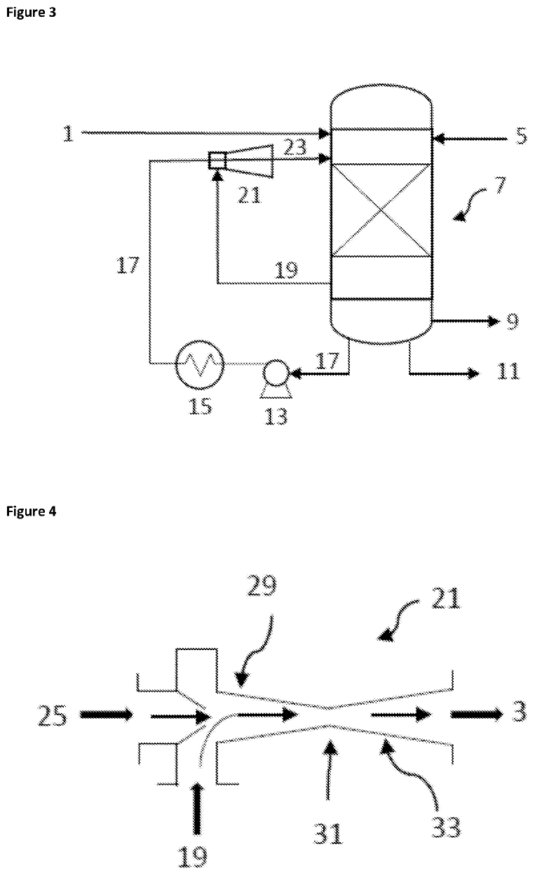

FIG. 3 illustrate a further process according to the invention. In this process, the motive fluid in the eductor is only liquid recycle stream 17. Accordingly, fresh substrate enters rector 7 independently via stream 1 and the combination of liquid recycle 17 and hydrogen gas recycle 19 enters the reactor 7 via stream 23.

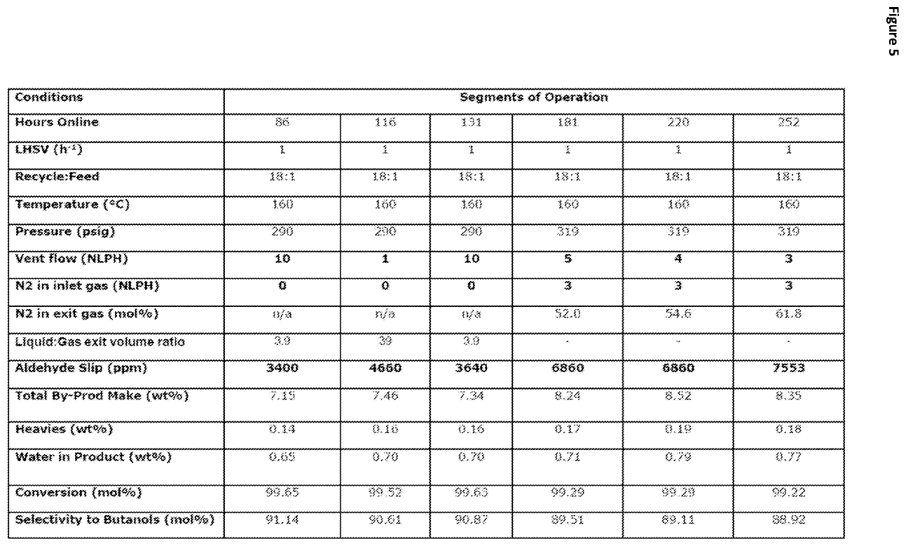

FIG. 4 illustrates an example of an eductor 21 which may be used in the present invention. The motive fluid 25 passes converging section 29 into throat 31 which causes a velocity increase and a pressure decrease. After exiting throat 31, the motive fluid enters the diverging nozzle 33, which causes a velocity decrease and a pressure increase creating a region of low pressure which draws hydrogen recycle stream 19 into the eductor. A combination of motive fluid and hydrogen recycle stream exits the eductor as single stream 3.

### EXAMPLES

The hydrogenation of butyraldehyde to produce n-butanol was carried out using an alumina supported copper hydrogenation catalyst in a test scale set-up representing the system shown in FIG. 1. The hydrogenation was carried out for 252 hours, with the specific conditions, products, selectivities and conversion of butyraldehyde shown in the table in FIG. 5. In summary, the reaction was run with a feed rate of 100 g/h, liquid recycle to feed ratio of 18:1, a liquid hourly space velocity (LHSV, h⁻¹) of 1.0, a catalyst bed exit temperature of 160° C., a pressure of 290 or 319 psig, varying vent flows of 1, 3, 4, 5 and 10 normal litres per hour (NLPH) and an inlet nitrogen gas flow of 0 and 3 NLPH. The flow of nitrogen gas serves to dilute the amount of hydrogen gas in the system and hence simulates lower hydrogen concentrations in a reactor. A volume ratio of liquid to gas exiting the reactor is provided for the run times in which pure hydrogen is used as the gas feed, which demonstrates the large hydrogen vent flow required to achieve a low volume ratio of liquid to gas exiting the reactor without a hydrogen gas recycle.

The data in the FIG. 5 table shows that having a greater flow of hydrogen results in an improved selectivity even though nominally the partial pressure of hydrogen exiting the reactor is the same. This is particularly evident by comparison of the data at 86 hours operation and 116 hours operation. When the vent is only 1 NLPH, giving close to stoichiometric levels of hydrogen in the reactor, the aldehyde slip and by-product make increases relative to when the vent is 10 NLPH and there are much greater than stoichiometric levels of hydrogen in the reactor, and there is a larger amount of available hydrogen in the reactor. Accordingly, when operating an industrial reactor at near stoichiometrically equivalent levels of hydrogen to minimise hydrogen loss (such as, e.g., the conditions at 116 hours in which the vent flow is 1 NLPH) in which the volume ratio of liquid exiting the reactor to gas exiting the reactor will typically be about 40:1 the efficiency of the hydrogenation reaction can be affected. The additional data points at 131, 181, 220 and 252 hours use nitrogen in the gas in order to simulate further hydrogen starvation in the reactor. It can be seen that reduced levels of available hydrogen in the reactor lead to even greater aldehyde slip and by-product make. The impact of very low exit gas volume is anticipated to be greater on an industrial plant with a significant diameter compared to a laboratory unit, for example due to the likelihood of any gas bubbles channelling rather than being evenly distributed.

The use of a hydrogen recycle stream in which the recycle energy of the liquid recycle is effectively used to pump the hydrogen recycle via an eductor, will reduce the volume ratio of liquid exiting the reactor to gas exiting the reactor, will increase the stoichiometric levels of hydrogen in the reactor and will increase the amount of hydrogen available for reaction. Thus it will provide the benefit shown by the data in Table 5. However, the flow of hydrogen into the reactor does not need to increase to increase available hydrogen and as such the vent flow does not need to be increased and a large stoichiometric excess of hydrogen does not need to be vented and lost from the system. Particularly, this is achieved without the need for an extra compressor because of the use of the recycle energy of the liquid product stream via the eductor. So, the extra cost and maintenance requirements associated with a mechanical compressor are not acquired.

A simulation of an eductor operating on an industrial process for hydrogenation of C9 aldehyde at an inlet pressure of 3.0 MPa with a motive fluid which is a liquid recycle comprising a combination of fresh aldehyde feed and liquid recycle running at 550,000 Kg/h, with a density of 730 Kg/m³, a viscosity of 0.5 mPas, a vapour pressure of 0.02 MPa, a motive pressure of 3.25 MPa and a temperature of 150° C., and a recycle gas running at 1040 Kg/hour, with a density of 3.0 Kg/m³, an eductor suction pressure of 2.9 MPa and a temperature of 160° C. reveals that exit liquid to exit gas ratio in the hydrogenation reactor can be 2.2:1 on a volume basis. Without the eductor the ratio of exit liquid to exit gas is around 80:1 on a volume basis. This shows that a beneficial flow of gas can be taken from the reactor for the recycle, with an operable pressure drop.

1. A process for hydrogenating a substrate in a hydrogenation reactor to provide a liquid product, said process comprising:
(a) supplying to the hydrogenation reactor;
(i) fresh substrate;
(ii) fresh hydrogen gas;
(ii) recycled liquid product;
(iii) recycled hydrogen gas;
(b) collecting liquid product in a liquid product stream from an outlet of the hydrogenation reactor;
(c) collecting liquid product in a liquid product recycle stream and returning at least portion to the hydrogenation reactor as the recycled liquid product;
(d) collecting hydrogen gas from an outlet of the hydrogenation reactor as a hydrogen gas recycle stream and returning at least a portion to the hydrogenation reactor as the recycled hydrogen gas;
wherein the hydrogen gas recycle stream is passed through an eductor before it is returned to the hydrogenation reactor; and
wherein the motive fluid in the eductor is the fresh substrate, the liquid product recycle stream or a combination of the fresh substrate and the liquid product recycle stream, or the motive fluid is the fresh hydrogen gas.

2. The process according to claim 1, wherein the substrate is an aldehyde and the product is an alcohol.

3. The process according to claim 2, wherein the aldehyde is an unsaturated aldehyde and the product is a saturated alcohol.

4. The process according to claim 1, wherein the motive fluid is a combination of the fresh substrate and the liquid product recycle stream.

5. The process according to claim 1, wherein the volume ratio of liquid exiting the reactor to gas exiting the reactor is less than 20:1.

6. The process according to claim 1, wherein the recycle ratio of liquid product to fresh substrate is greater than 10:1 on a mass basis.

7. The process according to claim 1, wherein the flow of hydrogen through the hydrogenation reactor is at least 120% of the stoichiometric flow of hydrogen required to hydrogenate the flow of substrate through the hydrogenation reactor.

8. The process according to claim 1, wherein the process further comprises a polishing reactor, and wherein hydrogen gas exiting the polishing reactor is combined with the hydrogen gas collected from the hydrogenation reactor to form part of the hydrogen gas recycle stream received by the eductor.

9. An apparatus for hydrogenating a substrate, said apparatus comprising;
(a) a hydrogenation reactor configured to hydrogenate the substrate and provide a liquid product;
(b) means for supplying fresh substrate to the hydrogenation reactor;
(c) means for supplying fresh hydrogen gas to the hydrogenation reactor;
(d) means for collecting a liquid product stream from an outlet of the hydrogenation reactor;
(e) means for collecting liquid product as a liquid product recycle stream;
(e) means for returning the liquid product recycle stream to the hydrogenation reactor;
(f) means for collecting hydrogen gas from an outlet of the hydrogenation reactor as a hydrogen gas recycle stream,
(g) means for returning the hydrogen gas recycle stream to the hydrogenation reactor;
(f) an eductor configured to receive the hydrogen gas recycle stream before it is returned to the hydrogenation reactor;
wherein the eductor is configured to use the fresh substrate, the liquid product recycle stream or a combination of the fresh substrate and the liquid product recycle stream as the motive fluid.
